(19) United States
(12) Patent Application Publication
(10) Pub. No.: US 2004/0209238 A1
(43) Pub. Date: Oct. 21, 2004

(54) Cell division-visualized cell and method of production of the same, method of detection of fluorescence, method of evaluation of influence upon cell division, and method of screenig

(75) Inventors: Kenji Sugimoto, Osaka-fu (JP); Takeshi Urano, Aichi-ken (JP); Makoto Tachibana, Kyoto-fu (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: NAGASE & CO., LTD., Osaka-shi (JP)

(21) Appl. No.: 10/718,712

(22) Filed: Nov. 24, 2003

(30) Foreign Application Priority Data
Dec. 9, 2002 (JP) 2002-357185

(51) Int. Cl. C12Q 1/00; C12N 5/00

(52) U.S. Cl. 435/4; 435/325

(57) ABSTRACT

The present invention relates to a cell division-visualized cell capable of visualizing cell division through incorporating fluorescent proteins into the cell, and a method of the production of the same, and a method of the visualization of cell division, a method of the evaluation of an influence upon cell division and a method of the screening using the same.

In the present invention, state of cell division is observed through visualizing cell division by (1) obtaining a fusion gene by allowing fusion of a gene of a protein that constitutes a cell structure which reflects the situation of cell division and a gene of a fluorescent protein, then (2) introducing three or more kinds of the aforementioned fusion genes of which fluorescent protein being the different kind into a host cell to obtain a cell division-visualized cell, and thereafter (3) allowing expression of the aforementioned fluorescent proteins to detect fluorescence derived from the aforementioned fluorescent proteins during cell division of the cell division-visualized cell in a time dependent manner. Further, by concurrently culturing a subject substance and the cell division-visualized cell of the invention, a subject substance that exerts an influence upon cell division can be selected.

### TECHNICAL FIELD

The present invention relates to a cell division-visualized cell capable of visualizing cell division through incorporating two or more kinds of fluorescent protein genes into the cell, and a method of the production of the same.

Further, the invention relates to a method of the detection of fluorescence in which expression of a fluorescent protein included in a cell division-visualized cell is allowed to detect the fluorescence derived from the fluorescent protein in a time dependent manner.

Moreover, the invention relates to a method of the evaluation of an influence upon cell division capable of evaluating the influence of a gene, an agent or the like upon cell division, through using the aforementioned cell division-visualized cell.

Additionally, the invention relates to a method of the screening in which a substance such as a gene or an agent that exerts an influence upon cell division is screened, through using the aforementioned cell division-visualized cell.

### BACKGROUND ART

Cells of animals and plants are alive while causing cell division, from their birth until death. The cell division cycle is composed of repetition of: a G1 phase (an intermediate phase occupying the time period between the mitosis and the stage of beginning of DNA synthesis), an S phase (a stage of DNA synthesis), a G2 phase (an intermediate phase occupying the time period between the S phase and mitosis) and an M phase (a stage from nuclear division to the initiation of actual cell division) in a time series order. It is often the case that one cycle of this cell division cycle takes about 8 to 24 hours, although the time period varies depending on the cell. In this cycle, the M phase (about 1 to 2 hours) is a cell division phase in which various structures within the cell vary in a most dynamical manner.

As described above, because cells of animals and plants are alive while causing cell division from their birth until death, it is of biologically important to observe the morphological alteration during cell division, in particular, to observe influences of any foreign substance, gene or the like upon morphological alteration during cell division. In this respect, morphological alteration during cell division has been conventionally observed. For example, substances that act on cell division generally exhibit a remarkable action upon the M phase during the aforementioned cell division cycle, therefore, chase of structural alteration during the M phase has been conduced through chasing the alteration of nucleus, chromosome, nuclear membrane, centrosome, centromere or spindle.

When alteration of the state of a cellular structure during cell division is observed, cells are visually observed using a microscope, in general. Because it is difficult to observe morphological alteration within a cell by general microscopic observation, observation by utilizing a fluorescent dye has been carried out heretofore. For example, a method has been executed in which cells are fixed with formalin, and observed after allowing a reaction thereto with an antibody or the like labeled with a fluorescent dye. According to this method, it is advantageous in that divided cells can be readily found.

However, when an influence of a subject substance upon cell division is evaluated, observation of cell division of a living cell would be desired. To the contrary, because cells are brought to death in the method described above, it is difficult to successively observe the morphological alteration of living cells although morphology of cells upon use of the fluorescent dye can be observed.

In such a respect, a method in which a fluorescent dye is introduced into living cells after binding to a protein has been also attempted.

However, in case of this method, the state of only the introduced cell can be observed, therefore, it is not easy to found a dividing cell. In addition, the protein bound to the fluorescent dye is decreased with every time of cell division, leading to a problem of the lowered fluorescence with time elapsed. Moreover, such a method still involves a problem of difficulty in observing a cell in just course of the division.

In such a respect, a method has been known in which nuclei or the like are labeled with one or two kinds of fluorescent protein(s), and the state of cell division is observed through the observation of this fluorescence (see, e.g., Sugimoto et al., “Cell Structure and Function” 25: 253-261 (2000), and Sugimoto et al., “Cell Structure and Function” 26: 705-718 (2001)).

However, when cell division is observed using one kind of a fluorescent protein, alteration of one kind of a protein (one kind of a cellular structure) can be merely determined, resulting in a problem of impossibility of determination of temporal and spatial movement among the cell structural components with each other. Furthermore, when cell division is observed using two kinds of fluorescent proteins, problems are involved in connection with difficulty to find spatial and mutual actions of various organelles during cell division due to small amount of the fluorescence (number of cellular structures). Moreover, as described above, too many invisible substances are present according to the method of observing cell division using one or two kinds of fluorescent protein(s), therefore, it is difficult to see details of the state of cell division. Accordingly, this method involves problems of impossibility to sufficiently discriminate when and how other substance, gene or the like exerts influences upon the cell division cycle.

### SUMMARY OF THE INVENTION

The present invention was made taking into account of the aforementioned circumstances, and an object of the invention is to provide a cell division-visualized cell capable of visualizing cell division through introducing a fluorescent protein into the cell, and a method of the production of the same.

Further, another object of the invention is to provide a method of the detection of fluorescence in which expression of a fluorescent protein included in a cell division-visualized cell is allowed to detect the fluorescence derived from the fluorescent protein in a time dependent manner.

Moreover, another object of the invention is to provide a method of the evaluation of an influence upon cell division capable of evaluating the influence of a gene, an agent or the like upon cell division, through using the aforementioned cell division-visualized cell.

Additionally, still another object of the invention is to provide a method of the screening in which a gene, an agent or the like that exerts an influence upon cell division is screened, through using the aforementioned cell division-visualized cell.

The present inventor studied taking into account of the aforementioned circumstances, and consequently found that fluorescence labeling may be performed as many as possible for a large variety of proteins that constitute a cell structure which reflects the situation of cell division. Thus, the inventor elaborately investigated, and consequently, succeeded in establishing a cell division-visualized cell, which is stable and does not eliminate a fluorescent protein even during the passage culture, through introducing three or more kinds of fusion genes including a fused protein that constitutes the target cell structure fused to a fluorescent protein, although no report has been heretofore published concerning a technique to dynamically visualize throughout the entire cell division through introducing three or more kinds of fluorescent proteins into a single cell. Then, by observing fluorescence using the cell division-visualized cell, cell division could be visualized in more detail than before. As a result, it was found that situation of the cell division and influences of other substance or gene upon cell division could be found in more detail. The present invention was thus accomplished.

The invention is as described below.

[1] A cell division-visualized cell which is obtained by the introduction into a host cell of three or more kinds of fusion genes obtained by allowing fusion of three or more kinds of genes of proteins that constitute a cell structure which reflects the situation of cell division, and genes of fluorescent proteins of the different kind, respectively.

[2] The cell division-visualized cell according to the above [1] wherein the aforementioned cell structure which reflects the situation of cell division is at least two kinds of nucleus, chromosome, nuclear membrane, centrosome, centromere, spindle, cytoskeleton, heterochromatin and telomere.

[3] The cell division-visualized cell according to the above [1] wherein the aforementioned protein that constitutes a cell structure which reflects the situation of cell division is at least two kinds of histone H3, histone H2B, importin a, lamin B, aurora A, aurora B, α-tubulin, β-tubulin, γ-tubulin, centromere protein A, centromere protein C, heterochromatin protein 1, survivin, actin, and a telomere protein.

[4] The cell division-visualized cell according to the above [1] wherein the aforementioned fluorescent proteins are two kinds or three or more kinds of green fluorescent proteins, cyan fluorescent proteins, red fluorescent proteins and yellow fluorescent proteins.

[5] The cell division-visualized cell according to the above [1] wherein the aforementioned host cell is a cell derived from a mammal.

[6] The cell division-visualized cell according to the above [5] wherein the aforementioned cell derived from a mammal is a somatic cell, a germ cell or an ES cell of a mammal.

[7] A cell division-visualized cell which is obtained by the introduction into a transformed cell obtained by the introduction into a host cell of two or more kinds of fusion genes that are obtained by allowing fusion of two or more kinds of genes of proteins that constitute a cell structure which reflects the situation of cell division and genes of fluorescent proteins of the different kind, respectively, of a fusion subject gene in which a subject gene is fused with a gene of a fluorescent protein of a different kind from that of the aforementioned fluorescent proteins.

[8] The cell division-visualized cell according to the above [7] wherein the aforementioned cell structure which reflects the situation of cell division is at least two kinds of nucleus, chromosome, nuclear membrane, centrosome, centromere, spindle, cytoskeleton, heterochromatin and telomere.

[9] The cell division-visualized cell according to the above [7] wherein the aforementioned protein that constitutes a cell structure which reflects the situation of cell division is at least two kinds of histone H3, histone H2B, importin a, lamin B, aurora A, aurora B, α-tubulin, β-tubulin, γ-tubulin, centromere protein A, centromere protein C, heterochromatin protein 1, survivin, actin, and a telomere protein.

[10] The cell division-visualized cell according to the above [7] wherein the aforementioned fluorescent proteins are two kinds or three or more kinds of green fluorescent proteins, cyan fluorescent proteins, red fluorescent proteins and yellow fluorescent proteins.

[11] A method of the production of a stable cell division-visualized cell which comprises:

(1) obtaining a fusion gene by allowing fusion of a gene of a protein that constitutes a cell structure which reflects the situation of cell division and a gene of a fluorescent protein, and then

(2) introducing three or more kinds of the aforementioned fusion genes of which fluorescent protein being the different kind into a host cell.

[12] A method of the production of a stable cell division-visualized cell which comprises:

(1) obtaining a fusion gene by allowing fusion of a gene of a protein that constitutes a cell structure which reflects the situation of cell division and a gene of a fluorescent protein, then

(2) introducing two or more kinds of the aforementioned fusion genes of which fluorescent protein being the different kind into a host cell to obtain a transformed cell, and thereafter

(3) introducing a fusion subject gene in which a subject gene is fused with a gene of a fluorescent protein of a different kind from that of the aforementioned fluorescent proteins, into the aforementioned transformed cell.

[13] A method of the detection of fluorescence which comprises:

(1) obtaining a fusion gene by allowing fusion of a gene of a protein that constitutes a cell structure which reflects the situation of cell division and a gene of a fluorescent protein, then

(2) introducing three or more kinds of the aforementioned fusion genes of which fluorescent protein being the different kind into a host cell to obtain a cell division-visualized cell, and thereafter

(3) allowing expression of the aforementioned fluorescent proteins to detect fluorescence derived from the aforementioned fluorescent proteins during cell division of the cell division-visualized cell in a time dependent manner.

[14] A method of the detection of fluorescence which comprises:

(1) obtaining a fusion gene by allowing fusion of a gene of a protein that constitutes a cell structure which reflects the situation of cell division and a gene of a fluorescent protein, then

(2) introducing two or more kinds of the aforementioned fusion genes of which fluorescent protein being the different kind into a host cell to obtain a transformed cell, thereafter

(3) introducing a fusion subject gene in which a subject gene is fused with a gene of a fluorescent protein of a different kind from that of the aforementioned fluorescent proteins, into the aforementioned transformed cell to obtain a cell division-visualized cell, and then

(4) allowing expression of the aforementioned fluorescent proteins to detect fluorescence derived from the aforementioned fluorescent proteins during cell division of the cell division-visualized cell, in a time dependent manner.

[15] A method of the evaluation of an influence upon cell division which comprises:

(1) culturing the cell division-visualized cell according to the above [1] in the presence of a subject substance, and then

(2) carrying out the observation of the state of cell division by detecting fluorescence generated through allowing expression of the aforementioned fluorescent proteins during cell division of the aforementioned cell division-visualized cell.

[16] A method of the evaluation of an influence upon cell division which comprises:

(1) culturing the cell division-visualized cell according to the above [7], and then

(2) carrying out the observation of the state of cell division by detecting fluorescence generated through allowing expression of the aforementioned fluorescent protein derived from the aforementioned fusion subject gene during cell division of the aforementioned cell division-visualized cell.

[17] The method of the evaluation of an influence upon cell division according to the above [15] wherein the aforementioned cell division is mitosis and/or meiosis.

[18] The method of the evaluation of an influence upon cell division according to the above [15] wherein the observation of the state of cell division is carried out by dynamic visualization through taking an image by chronological photographing of the aforementioned cell division-visualized cell during cell division under a fluorescence microscope or a laser microscope while culturing of the aforementioned cell division-visualized cell.

[19] A method of the screening which comprises selecting a subject substance which exerts an influence upon cell division by performing the method of the evaluation of an influence upon cell division according to the above [15].

[20] A method of the screening which comprises selecting a gene which exerts an influence upon cell division by performing the method of the evaluation of an influence upon cell division according to the above [16].

The cell division-visualized cell of the invention is a cell which stably expresses fluorescent proteins even during the passage culture, and fluorescence labeling is executed for a large variety of proteins that constitute a cell structure which reflects the situation of cell division. Therefore, the state of cell division can be observed in more detail than before through the observation of the fluorescence.

Other cell division-visualized cell of the invention is a stable cell which does not eliminate the fluorescent protein even during the passage culture. By observing the fluorescence, an influence of other gene to be evaluated upon cell division can be determined in detail.

When the aforementioned cell structure which reflects the situation of cell division is at least two kinds of nucleus, chromosome, nuclear membrane, centrosome, centromere, spindle, cytoskeleton, heterochromatin and telomere, visualization of alteration of the cell structure depending on the stage of the cell division is enabled, thereby allowing for observation of the state of cell division in more detail.

Further, when the aforementioned protein that constitutes a cell structure which reflects the situation of cell division is at least two kinds of histone H3, histone H2B, importin α, lamin B, aurora A, aurora B, α-tubulin, β-tubulin, γ-tubulin, centromere protein A, centromere protein C, heterochromatin protein 1, survivin, actin, and a telomere protein, visualization of alteration of the cell structure depending on the stage of the cell division is enabled, thereby allowing for observation of the state of cell division in more detail.

Moreover, when two kinds or three or more kinds of green fluorescent proteins, cyan fluorescent proteins, red fluorescent proteins and yellow fluorescent proteins are used as the aforementioned fluorescent proteins, fluorescence can be readily detected.

Further, when a cell derived from a mammal is used as the aforementioned cell into which the aforementioned fusion gene is introduced, the state of cell division in a mammal can be observed in more detail than before. In particular, when a somatic cell, a germ cell or an ES cell is used as the aforementioned cell derived from a mammal, the state of cell division in these cells can be observed in more detail than before.

According to the method of the production of a cell division-visualized cell of the invention and other method of the production of a cell division-visualized cell of the invention, the cell division-visualized cell exerting the effect as described above can be readily obtained.

According to the method of the detection of fluorescence, the state of cell division can be observed in more detail.

According to the method of the evaluation of an influence upon cell division of the invention and other method of the evaluation of an influence upon cell division of the invention, an influence of a subject substance or a subject gene upon cell division can be determined in detail.

Moreover, when the aforementioned cell division is mitosis and/or meiosis, an influence of a subject substance or a subject gene upon cell division can be determined in detail during such cell division.

Additionally, when the observation of the state of cell division is carried out by dynamic visualization through taking an image by chronological photographing of the aforementioned cell division-visualized cell during cell division under a fluorescence microscope or a laser microscope while culturing of the aforementioned cell division-visualized cell, an influence of a subject substance or a subject gene upon cell division, particularly time dependent alteration, can be determined in more detail.

According to the method of the screening of the invention and other method of the screening of the invention, a subject substance or a subject gene which exerts an influence upon cell division can be selected.

### DISCLOSURE OF THE INVENTION

The present invention is explained in detail below.

[1] Cell Division-Visualized Cell and Method of the Production of the Same

The cell division-visualized cell of the invention is obtained by the introduction into a host cell of three or more kinds of fusion genes obtained by allowing fusion of three or more kinds of genes of proteins that constitute a cell structure which reflects the situation of cell division, and genes of fluorescent proteins of the different kind, respectively. Because three or more kinds of proteins, which constitute a cell structure and which are fused with different fluorescent proteins, are present according to the cell division-visualized cell of the invention, three or more types of cell structures can be observed through fluorescence labeling. Consequently, the state of cell division can be observed in more detail compared to the observation through fluorescent labeling of one or two types of cell structure(s). In addition, dynamic observation of cell division is also enabled, thereby allowing for the understanding of time dependent alteration of each cell structure, and temporal and spatial correlation between respective cell structures can be also comprehended.

Other cell division-visualized cell of the invention is obtained by the introduction into a transformed cell obtained by the introduction into a host cell of two or more kinds of fusion genes that are obtained by allowing fusion of two or more kinds of genes of proteins that constitute a cell structure which reflects the situation of cell division and genes of fluorescent proteins of the different kind respectively, of a fusion subject gene in which a subject gene is fused with a gene of a fluorescent protein of a different kind from that of the aforementioned fluorescent proteins. According to the other cell division-visualized cell of the invention, fluorescence of three or more kinds of proteins in total, i.e., two or more kinds of proteins, which constitute a cell structure and which are fused with fluorescent proteins of the different kind, and a protein derived from a subject gene can be observed. As a consequence, an influence of the subject gene upon cell division can be observed in addition to the effect of observation of the cell division-visualized cell of the invention as described above.

The aforementioned cell structure which reflects the situation of cell division” is not limited for the type thereof as long as it constitutes a cell, and is a structural body which changes its shape and property during cell division (including a part of a structure that constitutes the structural body such as telomere which is a structure of a terminal end of an eucaryotic cell chromosome). Specific examples of the aforementioned cell structure that reflects the situation of the cell division include e.g., at least two (preferably three or more, and more preferably four or more) of nucleus, chromosome, nuclear membrane, centrosome, centromere, spindle, cytoskeleton, heterochromatin and telomere. Which cell structure should be selected as the aforementioned cell structure which reflects the situation of the cell division is not particularly limited,, however, it can be usually selected ad libitum on the basis of the stage of cell division to be observed. For example, when the observation at the prophase of the M phase is intended, nucleus (chromosome), nuclear membrane, centrosome, spindle, cytoskeleton, heterochromatin or telomere can be selected. Further, when the observation at the metaphase is intended, chromosome, centromere, spindle or heterochromatin can be selected. Moreover, when observation at the anaphase is intended, nucleus (chromosome), nuclear membrane or centromere can be selected.

The “protein that constitutes a cell structure which reflects the situation of cell division” is not particularly limited for the type thereof as long as it is a protein that constitutes a cell structure which reflects the situation of cell division as described above. Examples of the protein that constitutes a cell structure which reflects the situation of cell division include e.g., at least two (preferably three or more, and more preferably four or more) among the constitutive proteins of the aforementioned nucleus, chromosome, nuclear membrane, centrosome, centromere, spindle, cytoskeleton, heterochromatin and telomere. More specifically, examples thereof include e.g., at least two (preferably three or more, and more preferably four or more) of histone H3, histone H2B, importin α, lamin B, aurora A, aurora B, α-tubulin, β-tubulin, γ-tubulin, centromere protein A, centromere protein C, heterochromatin protein 1 (HP1α, HP1β or HP1γ), survivin, actin, and a telomere protein. In general, the cell structure which reflects the situation of the cell division described above to which visualization is expected, and the protein that constitutes the cell structure which reflects the situation of the cell division described above have correspondence as described below. According to the invention, among the proteins included in any of the following number (1) to (8), two or more, preferably three or more, and more preferably four or more proteins belonging to different number group can be selected.

(1) “nucleus/chromosome”: histone H3, histone H2B

(2) “nuclear membrane”: importin α, lamin B, nuclear lamin A precursor recognition factor (NARF)

(3) “centrosome”: aurora A, γ-tubulin

(4) “centrosome/spindle”: α-tubulin, β-tubulin, aurora A

(5) “heterochromatin” heterochromatin protein 1 (HP1α, HP1β or HP1γ), aurora B, survivin, SNF2b, (BRG1), Suv 39h1

(6) “cytoskeleton”: actin

(7) “telomere”: various types of telomere proteins (TRF1, TRF2 and the like)

(8) “centromere”: centromere protein A, C

Which protein should be selected as the aforementioned protein that constitutes a cell structure which reflects the situation of the cell division is not particularly limited, however, it can be usually selected ad libitum on the basis of the stage of cell division to which observation is intended. For example, the M phase includes the stage when the “nuclear membrane” disappears, the stage when the chromatin of the “nucleus” is condensed in the chromosome, and the stage when the “spindle” is formed, therefore, it is preferred that multiple proteins that constitute such cell structures are selected. More specifically, for example, for the period of from the G2 phase over the prophase of the M phase, two kinds or three or more kinds of proteins that constitute “nucleus/chromosome,” nuclear membrane” and “centrosome/spindle”, i.e., proteins separately belonging to the above (1) to (3) can be selected. Further, for the period of from the prometaphase, metaphase over the anaphase of the M phase, two kinds or three or more kinds of proteins that constitute “nucleus/chromosome”, “centrosome/spindle” and “centromere”, i.e., proteins separately belonging to the above (1), (3) and (8) can be selected. Moreover, for the period of from the anaphase over the telophase of the M phase, similarly to the period of from the G2 phase over the prophase of the M phase, proteins that constitute “nuclear membrane”, “nucleus/chromosome”, and “centrosome/spindle” can be selected.

The aforementioned “fluorescent protein” is not limited for the type thereof as long as it is expressed in a fused form with a protein that constitutes a cell structure or with a product of a subject gene at least during cell division of a cell division-visualized cell, and has a property to generate fluorescence. Examples of the fluorescent protein include one or two or more kinds of green fluorescent protein (GFP), cyan fluorescent protein (CFP), red fluorescent protein (DsRed, HcRed) and yellow fluorescent protein (YFP).

The “fusion gene” described above is obtained by allowing fusion of a gene of the aforementioned protein that constitutes a cell structure which reflects the situation of the cell division, with a gene of the aforementioned fluorescent protein. Process for obtaining the aforementioned fusion gene is not particularly limited. In general, it can be obtained by cleaving an expression vector comprising a gene of the fluorescent protein with suitable restriction enzymes; and inserting into the cleavage site a DNA fragment obtained by cleaving a gene of a protein that constitutes a cell structure which reflects the situation of the cell division with the same restriction enzymes as those described above. Alternatively, it may be obtained by allowing fusion of a cDNA of a protein that constitutes a cell structure which reflects the situation of the cell division with a gene of the fluorescent protein as described above.

According to the cell division-visualized cell of the invention, three or more kinds, preferably four or more kinds, and still more preferably five or more kinds of the fusion genes with the fluorescent protein of the different kind are introduced into a host cell. Further, according to other cell division-visualized cell of the invention, two or more kinds, preferably three or more kinds, and still more preferably four or more kinds of the fusion genes with the fluorescent protein of the different kind are introduced into a host cell. Process for introducing the fusion gene into the host cell is not particularly limited, but any known method can be selected as needed. Specific examples of the process for introducing the fusion gene into the host cell include e.g., electroporation method, transfection method, microinjection method and cell fusion methods such as protoplast fusion method, and the like. Also, the order of the introduction of the fusion genes into the host cell is not particularly limited. Furthermore, when the fusion gene is introduced into a host cell, each one kind of a fusion gene may be introduced into the host cell, or all the fusion genes may be introduced into the host cell together.

Additionally, the type of the host cell in not particularly limited, and any of a variety of cells can be used which allows observation of the cell division state. For example, the host cell may be a cell derived from a plant, or may be a cell derived from an animal. Exemplary animal cell which may be used is a cell derived from a mammal or a cell of a bird. When a cell derived from a mammal (e.g., human, mouse, rat, pig, calf, sheep and the like) is used as the host cell, the state of cell division in a mammal can be observed in more detail than before, and consequently, it is preferred because detailed evaluation is enabled on the influence of a substance upon cell division of a mammal, e.g., human. Furthermore, examples of the aforementioned host cell include e.g., somatic cells, germ cells and ES cells. More specifically, the examples include human MDA435 cell, mouse A9 cell, bovine MDBK cell, swine PK15, mouse C3H cell, Indian barking deer cell and the like.

Still more, according to other cell division-visualized cell of the invention, into a transformed cell obtained by the introduction of the aforementioned fusion gene into the host cell, is further introduced a fusion subject gene in which a subject gene is fused with a gene of a fluorescent protein of the different kind from that of the aforementioned fluorescent proteins. The subject gene may be a gene which is endogenous in the cell, or may be an exogenous gene. Alternatively, it may be a gene separated and extracted from a living body, or a gene fragment obtained by cleaving the gene with an appropriate restriction enzyme, or may be a synthetic polynucleotide chain. Also, process for introducing the fusion subject gene is not particularly limited, but any known method can be selected as needed, similarly to the process for introducing the aforementioned fusion gene.

In the method of the production of the cell division-visualized cell of the invention, process for selecting the intended stable cell division-visualized cell i.e., only the transformant including the introduced gene into the chromosome is not particularly limited. For example, the intended cell can be selected using an antibiotic such as puromycin.

[2] Method of Detection of Fluorescence

The method of the detection of fluorescence of the invention is characterized in that it comprises: (1) obtaining a fusion gene by allowing fusion of a gene of a protein that constitutes a cell structure which reflects the situation of cell division and a gene of a fluorescent protein, then (2) introducing three or more kinds of the aforementioned fusion genes of which fluorescent protein being the different kind into a host cell to obtain a cell division-visualized cell, and thereafter (3) allowing expression of the aforementioned fluorescent proteins to detect fluorescence derived from the aforementioned fluorescent proteins during cell division of the cell division-visualized cell in a time dependent manner.

Further, other method of the detection of fluorescence of the invention is characterized in that it comprises: (1) obtaining a fusion gene by allowing fusion of a gene of a protein that constitutes a cell structure which reflects the situation of cell division and a gene of a fluorescent protein, then (2) introducing two or more kinds of the aforementioned fusion genes of which fluorescent protein being the different kind into a host cell to obtain a transformed cell, thereafter (3) introducing a fusion subject gene in which a subject gene is fused with a gene of a fluorescent protein of the different kind from that of the aforementioned fluorescent proteins, into the aforementioned transformed cell to obtain a cell division-visualized cell, and then (4) allowing expression of the aforementioned fluorescent proteins to detect fluorescence derived from the aforementioned fluorescent proteins during cell division of the cell division-visualized cell in a time dependent manner.

In respect of the method of obtaining the aforementioned cell division-visualized cell and the context of the same, descriptions made hitherto for the cell division-visualized cell of the invention and for other cell division-visualized cell of the invention are applicable as they are.

In the method of the detection of fluorescence of the invention and other method of the detection of fluorescence of the invention, expression of the aforementioned fluorescent proteins is allowed for the purpose of enabling the observation of fluorescence derived from the fluorescent proteins at least during cell division of the cell division-visualized cell. In the method of the detection of fluorescence of the invention and other method of the detection of fluorescence of the invention, the aforementioned fluorescent proteins may be expressed for the purpose of enabling the observation of fluorescence derived from the fluorescent protein at least during cell division. In other words, expression of the fluorescent protein may be restricted only to the time period of cell division, or expression of the fluorescent protein may be allowed not only at the time period of cell division but also at a stage in which progress of the cell division is arrested. In addition, in the other method of the detection of fluorescence of the invention, the aforementioned fluorescent protein expressed for generating fluorescence may be expressed from the aforementioned fusion gene, or from the aforementioned fusion subject gene, or alternatively, may be expressed from both genes. Process for observing fluorescence derived from the aforementioned fluorescent protein is not particularly limited as long as generation of fluorescence derived from the fluorescent protein can be executed. For example, a process in which observation is carried out through generating fluorescence by a method of e.g., irradiation of a light such as an ultraviolet ray may be exemplified.

[3] Method of Evaluation of Influence Upon Cell Division

In the method of the evaluation of an influence upon cell division of the invention, observation of the state of cell division is carried out by (1) culturing the cell division-visualized cell of the invention or the other cell division-visualized cell of the invention in the presence of a subject substance, and then (2) detecting fluorescence generated through allowing expression of the aforementioned fluorescent proteins during cell division of the aforementioned cell division-visualized cell. According to the method of the evaluation of an influence upon cell division of the invention, an influence of a subject substance upon cell division can be evaluated in more detail than conventional methods.

The subject substance is not particularly limited for the kind thereof. The subject substance may be a substance derived from a natural product, or may be a synthetic substance. Specific examples of the subject substance include e.g., peptides, polypeptides, proteins, lipids, saccharides, microorganism fermentation products, extracts from an organism (including tissues of a plant or an animal, a microorganism, a cell or the like) (genes, polynucleotides and the like), synthetic compounds (low molecular weight organic compounds, high molecular weight organic compounds and the like), dioxin, endocrine disturbing chemicals, and libraries of the same. Examples of the library include synthetic compound libraries (combinatorial library and the like), peptide libraries (combinatorial library and the like) and the like. Further, the aforementioned subject substance which may be used is one kind alone, or two or more kinds (including a library or the like). Alternatively, the subject substance may be a composition comprising two or more kinds of substances, or may be a final product such as a pharmaceutical, food, cosmetic, pesticide or the like.

In the other method of the evaluation of an influence upon cell division of the invention, observation of the state of cell division is carried out by (1) culturing the other cell division-visualized cell of the invention, and then (2) detecting fluorescence generated through allowing expression of the aforementioned fluorescent protein derived from the aforementioned fusion subject gene during cell division of the aforementioned cell division-visualized cell.

The subject gene may be a gene which is endogenous in the cell, or may be an exogenous gene. Alternatively, it may be a gene separated and extracted from a living body, or a gene fragment obtained by cleaving such a gene with an appropriate restriction enzyme, or may be a synthetic DNA such as an antisense DNA or a PNA (peptide nucleic acid). Also, process for introducing the fusion subject gene is not particularly limited, but any known method can be selected as needed, similarly to the process for introducing the aforementioned fusion gene.

Conditions and methods of culture of the cell division-visualized cell of the invention or the other cell division-visualized cell of the invention are not particularly limited, but a variety of culture conditions and methods may be employed in compliance with various conditions such as type of the cell division-visualized cell.

In the method of the evaluation of an influence upon cell division of the invention, the state of cell division is observed by visualizing cell division through detecting fluorescence of the aforementioned fluorescent protein expressed from the aforementioned fusion gene during the cell division of the aforementioned cell division-visualized cell. Furthermore, in the other method of the evaluation of an influence upon cell division of the invention, the state of cell division is observed by visualizing cell division through detecting fluorescence of the aforementioned fluorescent protein expressed from the fusion subject gene. The observed cell division herein may be either mitosis or meiosis.

Process for detecting and observing fluorescence of the aforementioned fluorescent protein is not particularly limited. In general, fluorescence of the aforementioned fluorescent protein is directly observed with the eye using a fluorescence microscope or a laser microscope, or an image is obtained by photographing followed by carrying out the observation on the basis of the image. When the observation is carried out on the basis of the obtained image by photographing, images may be obtained by photographing for only a specified time period, however, dynamic visualization of cell division in enabled through taking images by photographing in a time dependent manner at predetermined time intervals. According to such a process, time dependent alteration of the cell division can be chased in more detail.

Moreover, the detection and observation of fluorescence of the aforementioned fluorescent protein may be carried out with varying conditions for observation such as observation time and the like ad libitum depending on type of the cell division-visualized cell, cell cycle to be observed, kind of the added subject substance, kind and properties of the added subject gene, and the like. Also, in instances of the aforementioned measurement by the dynamic visualization, time interval of the photographing may vary ad libitum depending on type of the cell division-visualized cell, cell cycle to be observed, kind of the added subject substance, kind and properties of the added subject gene, and the like.

[4] Method of Screening

The method of the screening of the invention is characterized in that a subject substance which exerts an influence upon cell division is selected by performing the method of the evaluation of an influence upon cell division of the invention as described above. An influence of a subject substance upon cell division can be evaluated in more detail than conventional methods by the method of the evaluation of an influence upon cell division of the invention, therefore, a substance that exerts an influence upon cell division can be selected in more segmentalized manner than conventional methods, according to the method of the screening of the invention. In other words, a substance or an agent which acts on a segmentalized phase of cell division can be selected.

In the method of the screening of the invention, the subject substance is not particularly limited for the kind thereof. The subject substance may be a substance derived from a natural product, or may be a synthetic substance. Specific examples of the subject substance include e.g., peptides, polypeptides, proteins, lipids, saccharides, microorganism fermentation products, extracts from an organism (including tissues of a plant or an animal, a microorganism, a cell or the like) (genes, polynucleotides and the like), synthetic compounds (low molecular weight organic compounds, high molecular weight organic compounds and the like), dioxin, endocrine disturbing chemicals, and libraries of the same. Examples of the library include synthetic compound libraries (combinatorial library and the like), peptide libraries (combinatorial library and the like) and the like. Further, the aforementioned subject substance which may be used is one kind alone, or two or more kinds (including a library or the like). For example, it is also possible that the screening according to the invention is carried out for a fraction of a mixture such as a cell extract, and a substance that exerts an influence upon cell division may be finally isolated through repeating fractionation. Alternatively, the subject substance may be a composition comprising two or more kinds of substances, or may be a final product such as a pharmaceutical, food, cosmetic, pesticide or the like.

The other method of the screening of the invention is characterized in that a gene which exerts an influence upon cell division is selected by performing the other method of the evaluation of an influence upon cell division of the invention. As described above, the other cell division-visualized cell of the invention can select a gene that exerts an influence upon cell division in more detail than before by fluorescent labeling of a protein that constitutes a cell structure which reflects the situation of the cell division, accompanied by introducing other gene into a cell and subjecting it to fluorescent labeling.

The aforementioned subject gene may be a gene which is endogenous in the cell, or may be an exogenous gene. Alternatively, it may be a gene separated and extracted from a living body, or a gene fragment obtained by cleaving such a gene with an appropriate restriction enzyme, or may be a synthetic DNA such as an antisense DNA or a PNA (peptide nucleic acid). Also, process for introducing the fusion subject gene is not particularly limited, but any known method can be selected as needed, similarly to the process for introducing the aforementioned fusion gene.

### BRIEF DESCRIPTION OF THE DRAWINGS

FIG. 1 is an explanatory drawing illustrating the state of cell division every two minutes in Example 1.

FIG. 2 is an explanatory drawing illustrating the state of cell division with time in Example 2.

FIG. 3 is an explanatory drawing illustrating the state of cell division with time of another cell in Example 2.

### BEST MODE FOR CARRYING OUT THE INVENTION

An example of the present invention is specifically explained below by way of Examples.

### EXAMPLE 1

(1) Production of Plasmid DNA

As an expression vector of a human aurora A-green fluorescent protein fusion protein, an expression vector for mammalian cells pEGFP-C1 (purchased from Clontech Co., Ltd.) which is a vector including a gene of a green fluorescent protein (GFP) was used. The pEGFP-C1 was cleaved with restriction enzymes SalI and BglII, and then a plasmid DNA (pEGFP-aurora A) for allowing the expression of a human aurora A-green fluorescent protein fusion protein was produced by inserting a 1.2 kb fragment of a human aurora cDNA into the cleaved site.

Further, as an expression vector of a histone H3-cyan fluorescent protein fusion protein, an expression vector for mammalian cells pECFP-C1 (purchased from Clontech Co., Ltd.) which is a vector including a gene of a cyan fluorescent protein (CFP) was used. Then, a mouse histone H3 expression plasmid pZErO-histone H3 (Tatchibana et al., J. Biol. Chem., Vol. 276, 25309-25317, 2001) was cleaved with restriction enzymes EcoRI and XhoI, and thus resulting 430 bp mouse histone H3 cDNA fragment was introduced into the cleaved site of pECFP-C1 which had been cleaved with the same restriction enzymes, thereby producing a plasmid DNA (pECFP-histone H3) for allowing the expression of a histone H3-cyan fluorescent protein fusion protein.

Moreover, as an expression vector of an importin α-red fluorescent protein fusion protein, an expression vector for mammalian cells pDsRed-C1 (purchased from Clontech Co., Ltd.) which is a vector including a gene of a red fluorescent protein (DsRed) was used. The pDsRed-C1 was cleaved with restriction enzymes EcoRI and SalI, and then a plasmid DNA (pDsRed-importin α) for allowing the expression of an importin a-red fluorescent protein fusion protein was obtained by inserting a 1.2 kb fragment of a human importin a cDNA into the cleaved site.

(2) Introduction of Plasmid DNA and Preparation of Stable Transformed Cell

As a cell to which the aforementioned plasmid DNA is introduced, human MDA435 cell (Vig et al., 1996; Sugimoto et al., 2000, 2001) was used. The human MDA435 cell was cultured in D-MEM medium containing 10% FCS (manufactured by Nissui Pharmaceutical Co., Ltd.) at 37° C. under 5% carbon dioxide in the air. Cells in the logarithmic growth phase were washed with 4 ml of 1×PBS (−), and the cells were detached by adding 1 ml of trypsin. After terminating the action of trypsin by adding 4 ml of D-MEM medium, the cell suspension was transferred into a centrifuge tube, and a part thereof was removed to subject to the measurement of cell number using a hematometer. The remaining part was centrifuged at 1000 rpm for 10 minutes, and thus recovered cells were washed by suspending in 12 ml of 1×PBS (−). This operation was repeated twice, and the cells were suspended in 1×K-PBS to give the cell density of 1.2×10⁷ cells/ml.

An aliquot of this suspension in an amount of 0.5 ml was transferred into a 1.5 ml microtube. After standing still for 5 minutes in ice, thereto was added 50 μl (corresponding to about 16 μg) of the aforementioned plasmid DNA (pEGFP-aurora A), and the mixture was gently stirred and left to stand still for 5 minutes in ice. After standing still, the cell suspension was stirred with a Pasteur pipette and transferred into a cuvette (manufactured by BIO-RAD) which had bee previously cooled. The aforementioned plasmid DNA (pEGFP-aurora A) was introduced into the human MDA435 cells by an electroporation method (power voltage: 0.22 kV, capacitance of capacitor: 960 μFD) using a pulse generator (“Gene Pulser”: manufactured by BIO-RAD), and the mixture was immediately left to stand still in ice for 10 minutes. Thereafter, thereto was added 0.5 ml of serum free D-MEM medium, and was stood still at room temperature for 10 minutes. The cells recovered with a Pasteur pipette were added to 4 ml of a medium, and the mixture of 0.3 to 0.5 ml each was seeded on 4 to 5 petri dishes (90 mm) in which 9 ml of a medium had been previously charged.

Two days following initiation of the culture, the medium was replaced with a medium including G418 (trade name “Geneticin”) added to give 0.8 to 1.2 mg/ml. The culture was continued while replacing the medium replaced every 5 days. After lapse of 2 to 3 weeks, each of thus formed colonies was subjected to a treatment with trypsin in a cloning ring having the inner diameter of 7 mm (manufactured by Iwaki Glass Co., Ltd.) independently. Each colony was transferred into a 12-well microplate, and was cultured for additional 5 to 10 days in 2 ml of a medium. Cells verified as expressing the intended protein at a ratio of 100% according to the operation described in the following paragraph [0049] were defined as stably transformed cell strain. The cells were further subjected to passage culture in a 90 mm dish, and stored.

Next, by the similar procedures as in the paragraphs [0045] and [0046], the aforementioned plasmid DNA (pECFP-histone H3) and the aforementioned plasmid DNA (pDsRed-importin α) were introduced. When the plasmid DNA (pDsRed-importin α) was introduced, 7 μg of a plasmid pTK-Hyg (hygromycin resistant plasmid) for a selection marker was further added, and on 1 to 2 days following introduction of the plasmid DNA into the cells, the cells were cultured in a medium including hygromycin that is an agent for selection added to give the concentration of 0.075 to 0.15 μg/ml. Thus, stably transformed cell strain was obtained according to the similar procedure as in the paragraph [0047]. Then, when the plasmid DNA (pECFP-histone H3) was introduced, 7 μg of a plasmid pLC-puro (puromycin resistant plasmid) for a selection marker was added, and on 1 to 2 days following introduction of the plasmid DNA into the cells, the cells were cultured in a medium including puromycin that is an agent for selection added to give the concentration of 0.05 to 0.5 μg/ml. Thus, stably transformed cell strain was obtained according to the similar procedure as described above.

(3) Process for Verification of Visualization of Target Structure in Transformed Cell

An aliquot (1×10⁵ to 3×10⁵) of the aforementioned transformed cells which formed a colony was further cultured on a cover glass, fixed with 4% paraformaldehyde for 20 minutes, and treated with 0.1% Triton X-100 for 5 minutes. Then, after subjecting the nuclei of thus fixed transformed cells to counter staining with a 1 μg solution of a blue fluorescent pigment DAPI, the transformed cells were observed using a fluorescence microscope “Eclipse E600” (manufactured by Nikon) equipped with a cooling CCD camera “MicroMAX 1300Y” (manufactured by Princeton Instruments Corporation), a controller “BioPoint MAC3000” (manufactured by Ludl Electric Products Ltd.) for controlling a filter wheel for excitation and a Z-axis motor, and an objective lens “PlanApo 60×” (NA1. 40, manufactured by Nikon), as described in Sugimoto et al., Cell Struct. Funct. Vol. 25, 253-261, 2000. Upon observation of each fluorescence, a filter set “Quad filter set No. 84” (manufactured by Chroma Technology Corp.) which allows for the observation of DAPI, CFP, GFP and DsRed was used. Upon image photographing and analysis, “MetaMorph software” (manufactured by Universal Imaging Corporation) was used, and the transformed cells were verified to express the intended fluorescent protein through observing that the target cell structure (centrosome/spindle, nucleus/chromosome, nuclear membrane) emitted fluorescence, respectively.

(4) Image Photographing of Intracellular Structure in Living Cell

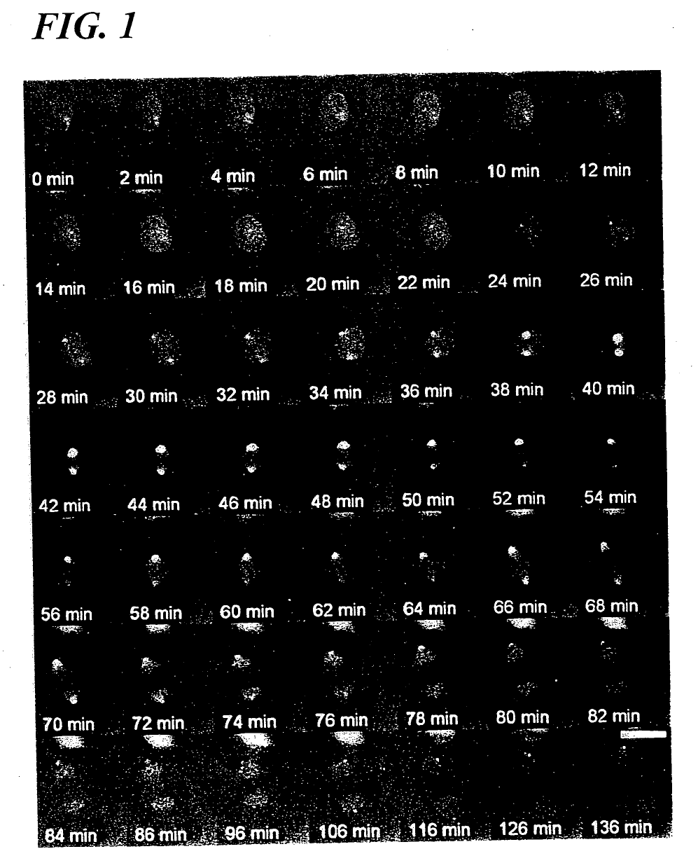

The transformed cells described above were cultured in a wet chamber with controlled carbon dioxide concentration and temperature by a thermostat and a control timer of carbon dioxide introduction (Japan, manufactured by Kokensya Engineering), using a 35 mm dish (with a cover glass attached on the bottom: manufactured by ASAHI TECHNO GLASS CORPORATION) on the stage of a inverted fluorescence microscope “Eclipse TE300” (manufactured by Nikon) equipped with a objective lens “PlanApo 60×”. Then, a highly sensitive CCD camera ORCA-ER (manufactured by Hamamatsu Photonics K.K.), and an excitation/absorption filter wheel equipped with a filter set for CFP/YFP/RFP for the observation (No. 86006, manufactured by Chroma Technology Corp.) and aZ-axis motor, and a controller “BioPoint MAC5000” (manufactured by Ludl Electronic Products Ltd.) which is a control equipment of the same were used, with the use of a computer software (LuminaVision version 1.40, manufactured by Mitani Co., Ltd.) for controlling these accessories. Upon observation at each time point, a light having a discrete wavelength per each fluorescent protein was irradiated in a sequential order, and the height of the stage (Z-axis) was altered with intervals of 1 to 2 μm for taking 4 to 10 images. Thus, 12 to 30 images in total were taken. This observation was continued for 120 to 180 minutes with the intervals of 2 minutes. Accordingly, approximately 1440 to 3600 images were obtained, and these were subjected to a time lapse analysis of the images using the aforementioned software. The results are shown in FIG. 1.

(5) Effects of Example 1

In FIG. 1, green fluorescence indicates the fluorescence of the aurora A-green fluorescent protein fusion protein; cyan fluorescence indicates the fluorescence of the histone H3-cyan fluorescent protein fusion protein; and red fluorescence indicates the fluorescence of the importin α-red fluorescent protein fusion protein. Accordingly, the centrosome/spindle (green fluorescence), the nuclei/chromosome (cyan fluorescence) and the nuclear membrane (red fluorescence) which are cell structures are visualized by these fusion proteins. In addition, the replicated centrosomes (green fluorescence) were present on an approximately identical position for 0 to 12 minutes following initiation of the observation, however, they started to move to achieve division at 14 minutes following initiation of the observation. At approximately 30 minutes following initiation of the observation, the centrosomes (green fluorescence) completed the movement to both poles, respectively. This period corresponds to the later stage of the G2 phase of the cell division cycle. From this result, discrimination between the G2 phase and the prophase of the M phase, which was not necessarily clarified by usual microscopic examination, could be distinctly captured as a period when centrosomes that were visualized by aurora A move to both poles.

Then, since 30 minutes past following initiation of the observation, condensation and movement of the nuclear chromatin (cyan fluorescence) toward the nuclear membrane started. Further, thickening accompanied by recess of the nuclear membrane (red fluorescence) at a part immediately below the centrosome (green fluorescence) started, and at 44 minutes following initiation of the observation, red fluorescence vanished suggesting that the nuclear membrane disappeared. On the other hand, it is proven that the centrosome (green fluorescence) rapidly grew since 38 minutes following initiation of the observation when the nuclear membrane was disrupted, and deformed into the shape like a spindle at 44 minutes following initiation of the observation. This time period corresponds to the prophase of the M phase of the cell division cycle. Accordingly, structural alteration from the prophase over the prometaphase in the M phase could be captured.

Next, from 44 minutes over 64 minutes following initiation of the observation, alignment of the chromosomes (cyan fluorescence) in a direction of equatorial line of the spindle (green fluorescence) was observed together with the formation of a spindle (green fluorescence). This time period corresponds to from the prometaphase to the metaphase of the M phase of the cell division cycle. Accordingly, structural alteration from the prometaphase over the metaphase in the M phase could be captured.

Then, from 66 minutes following initiation of the observation, the chromosomes (cyan fluorescence) moved toward the direction of poles, and they were observed to reach to both poles at 70 minutes following initiation of the observation. Accordingly, the stage when the anaphase of the M phase, in particular the anaphase of the M phase, is initiated could be more clearly captured.

Next, it was observed that the nuclear membrane (red fluorescence) arose again such that it encompasses the chromosomes (cyan fluorescence), which had been distributed to both poles, since 72 minutes following initiation of the observation. Accordingly, discrimination between the anaphase and telophase of the M phase was clarified. Thereafter, the spindle (green fluorescence) was condensed, and it was ascertained to be present as a centrosome in the vicinity of the nuclear membrane at 96 minutes following initiation of the observation. In addition, it was ascertained that the chromosomes (cyan fluorescence) were gradually discondensed since 72 minutes following initiation of the observation, thereby enlarging the size of the nuclei. From these results of observation, transition from the telophase to the G1 phase is revealed.

Accordingly, by observing fluorescence using the cell division-visualized cell of the invention, temporal and spatial structural alteration of the nuclear membrane visualized with importin α, the nuclei and chromosome visualized with histone H3, and the centrosome and spindle visualized with aurora A in a living cell could be dynamically observed. Hence, discrimination between the G2 phase and the prophase, initiation of the anaphase, and discrimination between the anaphase and the telophase, which were not apparently determined by conventional microscopic examination which had been generally performed could be thereby clarified. More specifically, the G2 phase, which was generally hard to be captured distinctly, could be distinctly captured by visualizing centrosomes (corresponding to green fluorescence) as a stage in which these are divided into two sides (12 to 30 minutes in FIG. 1). Also, transition of from the G2 phase to the prophase of the M phase can be captured as a stage in which the nuclear membrane (corresponding to red fluorescence) below the centrosome thickens and begins to be recessed (32 minutes in FIG. 1). Moreover, the prophase over the prometaphase of the M phase could be captured as a stage in which the recessed nuclear membrane gradually diminishes and the centrosomes grow (40 to 44 minutes in FIG. 1); the transition of from the metaphase to the anaphase could be captured as a stage in which the chromosomes (corresponding to blue fluorescence) start to divide (66 minutes); and the transition of from the anaphase to the telophase could be captured as a stage in which the nuclear membrane is reformed (72 minutes). These clearly suggest that observation of the fluorescence using the cell division-visualized cell of the invention enables the observation of the process of the cell division in more detail than before.

### EXAMPLE 2

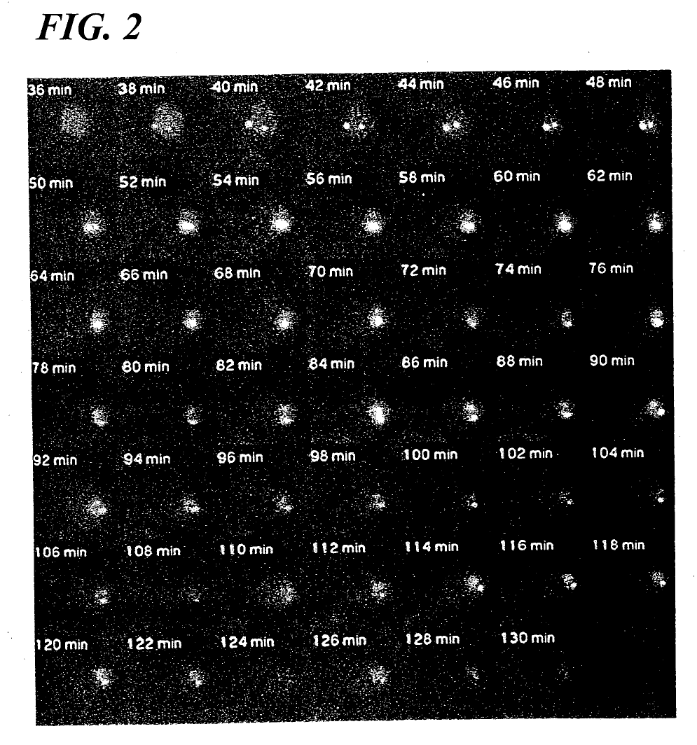

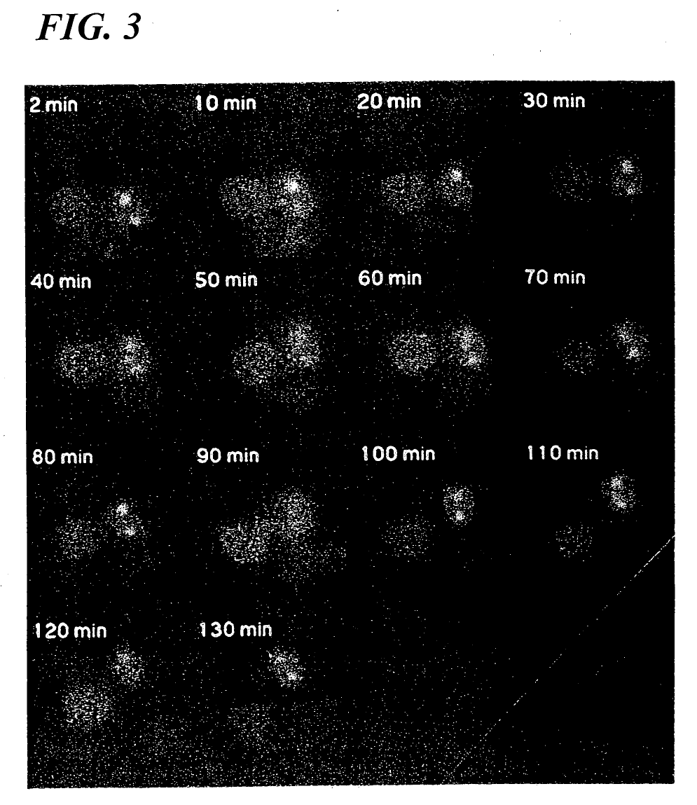

This Example observes the action of an inhibitory substance of cell division against cell division in real time. The aforementioned transformed cell obtained in Example 1 was cultured using a 35 mm dish (with a cover glass attached on the bottom: manufactured by ASAHI TECHNO GLASS CORPORATION) in a wet chamber with controlled carbon dioxide concentration and temperature. Next, to the culture fluid was added vinblastine as an agent, and the dynamic alteration of the visualized cell structure was observed under an inverted fluorescence microscope manufactured by Nikon with the intervals of 2 minutes, using a highly sensitive CCD camera ORCA-ER (manufactured by Hamamatsu Photonics K.K.), and an excitation/absorption filter wheel equipped with a filter set for CFP/YFP/RFP (No. 86006, manufactured by Chroma Technology Corp.) and a Z-axis motor, and a controller “BioPoint MAC5000” (manufactured by Ludl Electronic Products Ltd.) which is a control equipment of the same, with the use of a computer software (LuminaVision, manufactured by Mitani Co., Ltd.) for controlling these accessories. Upon observation at each time point, a light having a discrete wavelength per each fluorescent protein was irradiated in a sequential order, and the height of the stage (Z-axis) was altered with intervals of 2 μm for taking 18 images in total. The results are shown in FIG. 2 and FIG. 3.

As is shown in FIG. 2, through the addition of vinblastine, growing of centrosomes (green fluorescence) which is characteristic in the prophase was observed from 38 minutes following initiation of the observation, however, inhibition was made on the movement of the centrosomes toward the poles which should occur in the cell from the prophase over prometaphase (note that they contrarily approximated at 48 minutes following the initiation) and on the formation of the spindle. In addition, also the alignment of the chromosomes on the equatorial plane which should occur thereafter, i.e., transition to the metaphase, and the following distribution of the chromosomes to both poles (transition to the anaphase) were not found even after lapse of 130 minutes following initiation of the observation. From these results, inhibition of the progress of cell division, particularly inhibition of the transition from the prophase to the prometaphase by vinblastine could be observed.

Moreover, as shown in FIG. 3, inhibition of the transition from the prophase to the prometaphase by addition of vinblastine was observed also in other cell, and the alignment of the chromosomes on the equatorial plane which should be found at the metaphase was not observed even after lapse of 130 minutes. These results further support the inhibition of the progress of cell division, particularly inhibition of the transition from the prophase to the prometaphase by addition of vinblastine.

The present invention is not limited to the specific Examples as described above, but may involve Examples with various modifications depending on the object and application; within the scope of the invention.

Industrial Applicability

According to the present invention, the state of cell division can be observed in more detail than before because fluorescent proteins are stably expressed even during the passage culture, and fluorescence labeling is executed for a large variety of proteins that constitute a cell structure which reflects the situation of cell division. The invention can be suitably used for applications of selection of a subject substance or a subject gene which exerts an influence upon cell division, and the like, in addition to the observation of cell division.

1. A cell division-visualized cell which is obtained by the introduction into a host cell of three or more kinds of fusion genes obtained by allowing fusion of three or more kinds of genes of proteins that constitute a cell structure which reflects the situation of cell division, and genes of fluorescent proteins of the different kind, respectively.

2. The cell division-visualized cell according to claim 1 wherein said cell structure which reflects the situation of cell division is at least two kinds of nucleus, chromosome, nuclear membrane, centrosome, centromere, spindle, cytoskeleton, heterochromatin and telomere.

3. The cell division-visualized cell according to claim 1 wherein said protein that constitutes a cell structure which reflects the situation of cell division is at least two kinds of histone H3, histone H2B, importin α, lamin B, aurora A, aurora B, α-tubulin, β-tubulin, γ-tubulin, centromere protein A, centromere protein C, heterochromatin protein 1, survivin, actin, and a telomere protein.

4. The cell division-visualized cell according to claim 1 wherein said fluorescent proteins are two kinds or three or more kinds of green fluorescent proteins, cyan fluorescent proteins, red fluorescent proteins and yellow fluorescent proteins.

5. The cell division-visualized cell according to claim 1 wherein said host cell is a cell derived from a mammal.

6. The cell division-visualized cell according to claim 5 wherein said cell derived from a mammal is a somatic cell, a germ cell or an ES cell of a mammal.

7. A cell division-visualized cell which is obtained by the introduction into a transformed cell obtained by the introduction into a host cell of two or more kinds of fusion genes that are obtained by allowing fusion of two or more kinds of genes of proteins that constitute a cell structure which reflects the situation of cell division and genes of fluorescent proteins of the different kind, respectively, of a fusion subject gene in which a subject gene is fused with a gene of a fluorescent protein of a different kind from that of said fluorescent proteins.

8. The cell division-visualized cell according to claim 7 wherein said cell structure which reflects the situation of cell division is at least two kinds of nucleus, chromosome, nuclear membrane, centrosome, centromere, spindle, cytoskeleton, heterochromatin and telomere.

9. The cell division-visualized cell according to claim 7 wherein said protein that constitutes a cell structure which reflects the situation of cell division is at least two kinds of histone H3, histone H2B, importin α, lamin B, aurora A, aurora B, α-tubulin, β-tubulin, γ-tubulin, centromere protein A, centromere protein C, heterochromatin protein 1, survivin, actin, and a telomere protein.

10. The cell division-visualized cell according to claim 7 wherein said fluorescent proteins are two kinds or three or more kinds of green fluorescent proteins, cyan fluorescent proteins, red fluorescent proteins and yellow fluorescent proteins.

11. A method of the production of a stable cell division-visualized cell which comprises:
(1) obtaining a fusion gene by allowing fusion of a gene of a protein that constitutes a cell structure which reflects the situation of cell division and a gene of a fluorescent protein, and then
(2) introducing three or more kinds of said fusion genes of which fluorescent protein being the different kind into a host cell.

12. A method of the production of a stable cell division-visualized cell which comprises:
(1) obtaining a fusion gene by allowing fusion of a gene of a protein that constitutes a cell structure which reflects the situation of cell division and a gene of a fluorescent protein, then
(2) introducing two or more kinds of said fusion genes of which fluorescent protein being the different kind into a host cell to obtain a transformed cell, and thereafter
(3) introducing a fusion subject gene in which a subject gene is fused with a gene of a fluorescent protein of a different kind from that of said fluorescent proteins, into said transformed cell.

13. A method of the detection of fluorescence which comprises:
(1) obtaining a fusion gene by allowing fusion of a gene of a protein that constitutes a cell structure which reflects the situation of cell division and a gene of a fluorescent protein, then
(2) introducing three or more kinds of said fusion genes of which fluorescent protein being the different kind into a host cell to obtain a cell division-visualized cell, and thereafter
(3) allowing expression of said fluorescent proteins to detect fluorescence derived from said fluorescent proteins during cell division of said cell division-visualized cell in a time dependent manner.

14. A method of the detection of fluorescence which comprises:
(1) obtaining a fusion gene by allowing fusion of a gene of a protein that constitutes a cell structure which reflects the situation of cell division and a gene of a fluorescent protein, then
(2) introducing two or more kinds of said fusion genes of which fluorescent protein being the different kind into a host cell to obtain a transformed cell, thereafter
(3) introducing a fusion subject gene in which a subject gene is fused with a gene of a fluorescent protein of a different kind from that of said fluorescent proteins, into said transformed cell to obtain a cell division-visualized cell, and then
(4) allowing expression of said fluorescent proteins to detect fluorescence derived from said fluorescent proteins during cell division of said cell division-visualized cell, in a time dependent manner.

15. A method of the evaluation of an influence upon cell division which comprises:
(1) culturing the cell division-visualized cell according to claim 1 in the presence of a subject substance, and then
(2) carrying out the observation of the state of cell division by detecting fluorescence generated through allowing expression of said fluorescent proteins during cell division of said cell division-visualized cell.

16. A method of the evaluation of an influence upon cell division which comprises:
(1) culturing the cell division-visualized cell according to claim 7, and then
(2) carrying out the observation of the state of cell division by detecting fluorescence generated through allowing expression of said fluorescent protein derived from said fusion subject gene during cell division of said cell division-visualized cell.

17. The method of the evaluation of an influence upon cell division according to claim 15 wherein said cell division is mitosis and/or meiosis.

18. The method of the evaluation of an influence upon cell division according to claim 15 wherein the observation of the state of cell division is carried out by dynamic visualization through taking an image by chronological photographing of said cell division-visualized cell during cell division under a fluorescence microscope or a laser microscope while culturing of said cell division-visualized cell.

19. A method of the screening which comprises selecting a subject substance which exerts an influence upon cell division by performing the method of the evaluation of an influence upon cell division according to claim 15.

20. A method of the screening which comprises selecting a gene which exerts an influence upon cell division by performing the method of the evaluation of an influence upon cell division according to claim 16.
